(19) United States
(12) Patent Application Publication
(10) Pub. No.: US 2026/0077013 A1
(43) Pub. Date: Mar. 19, 2026

(54) COMBINATIONS FOR USE IN CANCER THERAPY

(71) Applicant: Lisata Therapeutics, Inc., Basking Ridge, NJ (US)

(72) Inventor: Andrew Dean, Basking Ridge, NJ (US)

(21) Appl. No.: 19/328,138

(22) Filed: Sep. 13, 2025

Related U.S. Application Data
(60) Provisional application No. 63/694,679, filed on Sep. 13, 2024.

(51) Int. Cl. A61K 38/12; A61K 31/337; A61K 31/7068; A61K 39/00; A61K 39/395; A61P 35/00

(52) U.S. Cl. CPC A61K 38/12; A61K 31/337; A61K 31/7068; A61K 39/3955; A61P 35/00; A61K 2039/505

(57) ABSTRACT

Provided herein are methods, compositions and medicaments for treating solid tumor cancers, including pancreatic cancers.

### CROSS-REFERENCE TO RELATED APPLICATIONS

This application claims priority to U.S. Provisional Patent Application Ser. No. 63/694,679, filed on Sep. 13, 2024, the contents of which is hereby incorporated by reference in its entirety into this application.

### BACKGROUND

The invention relates to methods and medicaments useful for treating solid tumors.

The National Cancer Institute estimates that in 2018 approximately 1,735,350 new cases of cancer will be diagnosed in the United States and 609,640 people will die from the disease. Despite advances in the treatment of certain forms of cancer through surgery, radiotherapy, chemotherapy, and most recently immunotherapy, most types of solid tumors are essentially incurable. Even when an effective treatment is available for a particular cancer, the side effects from the treatment can have a significant adverse impact on a patient's quality of life.

Pancreatic cancer is an especially serious cancer and a life-threatening condition. In most cases, early stages of the disease are asymptomatic and less than 20% of pancreatic cancers are amenable to surgery. Moreover, invasive and metastatic pancreatic cancers respond poorly to existing treatments in chemotherapy and radiotherapy, with response rates typically less than 30%. The National Cancer Institute (NCI) estimate that survival rate for cancer of the exocrine pancreas is less than 5% and the median survival time after diagnosis is less than a year. The continuing poor prognosis and lack of effective treatments for pancreatic cancer highlight an unmet medical need to develop less toxic and more efficient treatment strategies that improve the clinical management and prognosis of patients afflicted with pancreatic cancer.

An important reason for why most anti-cancer agents have toxicity and limited efficacy for solid tumors is the fact that anti-cancer drugs only penetrate 3-5 cell diameters deep from the blood vessels, leaving some areas of the tumor exposed to an ineffective concentration of the drug or to no drug at all. For example, studies have suggested that less than 1% of the administered nabpaclitaxel may be able to penetrate/enter the pancreatic ductal adenocarcinoma tissue.

### SUMMARY

Accordingly, provided herein are compositions and methods of treating pancreatic cancer in a patient in need thereof, comprising administering to the patient an effective amount of LSTA-1, or pharmaceutically acceptable salt thereof, in combination with anti-cancer therapy comprising durvalumab, gemcitabine and nab-paclitaxel, or pharmaceutically acceptable salts thereof. In particular embodiments, the pancreatic cancer is selected from the group consisting of: primary pancreatic cancer, metastatic pancreatic cancer, refractory pancreatic cancer, cancer drug resistant pancreatic cancer and pancreatic ductal adenocarcinoma. In a particular embodiment, the cancer is pancreatic ductal adenocarcinoma (Stage 0-IV).

The clinical trial results provided herein indicate that combining LSTA-1 (certepeptide), an iRGD-analog, with an anti-cancer therapy comprising durvalumab, gemcitabine, and nab-paclitaxel significantly increases the tumor penetration of these drugs and improves their efficacy.

Accordingly, also provided herein are methods of treating or preventing Alzheimer's disease.

The numbered items below describe these methods and compositions:

1. A method of treating pancreatic cancer in a patient in need thereof, comprising administering to the patient an effective amount of LSTA-1, or pharmaceutically acceptable salt thereof, in combination with anti-cancer therapy comprising durvalumab, gemcitabine and nab-paclitaxel, or pharmaceutically acceptable salts thereof.

2. The method of item 1, wherein the pancreatic cancer is selected from the group consisting of: primary pancreatic cancer, metastatic pancreatic cancer, refractory pancreatic cancer, cancer drug resistant pancreatic cancer and pancreatic ductal adenocarcinoma (PDAC).

3. The method of items 1-2, wherein the cancer is pancreatic ductal adenocarcinoma (Stage 0-IV).

4. The method of items 1-3, wherein LSTA-1 is administered in an amount selected from the group consisting of: about 0.2 to 20 mg/kg body weight/per dose of anti-cancer therapy, about 0.3 to 17 mg/kg body weight/per dose of anti-cancer therapy, about 0.4 to 14 mg/kg body weight/per dose of anti-cancer therapy, about 0.5 to 11 mg/kg body weight/per dose of anti-cancer therapy, about 0.6 to 8 mg/kg body weight/per dose of anti-cancer therapy, about 0.7 to 5 mg/kg body weight/per dose of anti-cancer therapy, about 0.8 to 3.2 mg/kg body weight/per dose of anti-cancer therapy.

5. The method of items 1-4, wherein LSTA-1 is administered in an amount corresponding to 3.2 mg/kg body weight/per dose.

6. The method of items 1-5, wherein LSTA-1 is administered before or during the administration of anti-cancer therapy, wherein the anti-cancer therapy is at a dosing regimen selected from the group consisting of: 4 times/day, 3 times/day, twice daily, once daily, once every other day, once every 2nd day, once every 3rd day, once every 4th day, once every 5th day, once every 6th day, once weekly, once every 8th day, once every 9th day, once every 10th day, once every 11th day, once every 12th day, once every 13th day, once every 2 weeks, once every 3 weeks, and once per month.

7. The method of items 1-6, wherein:
LSTA-1 is administered in a range amount selected from: 0.01-100, 0.02-90, 0.03-80, 0.04-70, 0.05-60, 0.06-50, 0.07-40, 0.08-30, 0.09-30, 0.1-25, 0.11-20, 0.12-15, 0.13-10, 0.14-9, 0.15-8, 0.16-7, 0.17-6, 0.18-5, 0.19-4, and 0.2-3.2 mg/kg body weight/day or per dose;
durvalumab is administered in a range amount selected from: 1-2000, 50-1900, 100-1800, 200-1700, 300-1600, 350-1500, 400-1400, 450-1300, 500-1200, 550-1100, 600-1000, 650-900, and 700-800 mg;
nab-paclitaxel is administered in a range amount selected from: 1-500, 10-450, 20-400, 30-350, 40-300, 50-250, 60-200, 70-175, 80-160, 90-150, 100-140, 110-140, 115-135 and 120-130 mg/m²; and
gemcitabine is administered in a range amount selected from: 1-5000, 100-4500, 200-4000, 300-3500, 400-3000, 500-2500, 550-2000, 600-1750, 650-1500, 700-1400, 750-1300, 800-1200, and 900-1100 mg/m².

8. The method of items 1-7, wherein LSTA-1 is administered at 3.2 mg/kg body weight/day or per dose; durvalumab is administered at 750 mg; nab-paclitaxel is administered at 125 mg/m²; and gemcitabine is administered at 1000 mg/m².

9. The method of items 1-8, wherein LSTA-1 (certepeptide) is administered at 3.2 mg/kg body weight/day or per dose on days 1, 2, 8, 15, 16 in 28-day cycles; durvalumab is administered at 750 mg on days 1 and 15 in 28-day cycles; nab-paclitaxel is administered at 125 mg/m² on days 1, 8, 15 in 28-day cycles; and gemcitabine is administered at 1000 mg/m² on days 1, 8, 15 in 28-day cycles.

10. The method of item 9, wherein the dosing regimen is administered for at least 2 cycles, at least 4 cycles, at least 6 cycles, at least 8 cycles, at least 10 cycles of administration; or for: 2 cycles, 4 cycles, 6 cycles, 8 cycles, 10 cycles, or 12 cycles of administration.

11. The method of item 10, the dosing regimen is administered for 6 cycles.

12. The method of items 1-11, wherein efficacy or clinical activity of the method is measured by determining RECIST response, wherein after 6 treatment cycles: 12/20 patients demonstrated partial response; 7 patients demonstrated stable disease; and 1 patient demonstrated complete response.

13. The method of items 1-12, wherein efficacy or clinical activity of the method is measured by determining CA19-9 levels, wherein after 6 treatment cycles: 15/20 patients demonstrated a decrease in CA19-9 levels, and 8 of these 15 patients demonstrated >90% decrease in CA19-9 levels.

14. The method of items 1-13, wherein efficacy or clinical activity of the method is measured by determining infiltration of TILs into tumor stroma, wherein wherein after 6 treatment cycles: 9/10 patients biopsied demonstrated 15%-50% stroma infiltration by TILs.

15. A method for treating, inhibiting, or reducing the volume of a tumor of a cancer in a subject or patient in need thereof, wherein the method comprises administering a dosing regimen of an iRGD, or a pharmaceutically acceptable salt thereof, in a combination with simultaneous, separate or sequential administration of an anti-cancer therapy comprising an immune checkpoint inhibitor, a nucleoside; and a taxane.

16. The method of item 15, wherein:
iRGD is administered in a range amount selected from: 0.01-100, 0.02-90, 0.03-80, 0.04-70, 0.05-60, 0.06-50, 0.07-40, 0.08-30, 0.09-30, 0.1-25, 0.11-20, 0.12-15, 0.13-10, 0.14-9, 0.15-8, 0.16-7, 0.17-6, 0.18-5, 0.19-4, and 0.2-3.2 mg/kg body weight/day or per dose;
the immune checkpoint inhibitor is administered in a range amount selected from: 1-2000, 50-1900, 100-1800, 200-1700, 300-1600, 350-1500, 400-1400, 450-1300, 500-1200, 550-1100, 600-1000, 650-900, and 700-800 mg;
the taxane is administered in a range amount selected from: 1-500, 10-450, 20-400, 30-350, 40-300, 50-250, 60-200, 70-175, 80-160, 90-150, 100-140, 110-140, 115-135 and 120-130 mg/m²; and
the nucleoside is administered in a range amount selected from: 1-5000, 100-4500, 200-4000, 300-3500, 400-3000, 500-2500, 550-2000, 600-1750, 650-1500, 700-1400, 750-1300, 800-1200, and 900-1100 mg/m².

17. The method of items 15-16, wherein: the iRGD is administered at 3.2 mg/kg body weight/day or per dose; the immune checkpoint inhibitor is administered at 750 mg; the taxanel is administered at 125 mg/m²; and the nucleoside is administered at 1000 mg/m².

18. The method of items 15-17, wherein the iRGD is LSTA-1 (certepeptide) administered at 3.2 mg/kg body weight/day or per dose on days 1, 2, 8, 15, 16 in 28-day cycles; wherein the immune checkpoint inhibitor is durvalumab administered at 750 mg on days 1 and 15 in 28-day cycles; the taxanel is nab-paclitaxel administered at 125 mg/m² on days 1, 8, 15 in 28-day cycles; and wherein the nucleoside is gemcitabine administered at 1000 mg/m² on days 1, 8, 15 in 28-day cycles.

19. The method of item 18, wherein the dosing regimen is administered for at least 2 cycles, at least 4 cycles, at least 6 cycles, at least 8 cycles, at least 10 cycles of administration; or for: 2 cycles, 4 cycles, 6 cycles, 8 cycles, 10 cycles, or 12 cycles of administration.

20. The method of item 19, wherein the dosing regimen is administered for 6 cycles.

21. The method of items 15-20, wherein the tumor is a solid tumor of a cancer selected from the group consisting of: breast cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colon cancer, colorectal cancer, endometrial carcinoma, salivary gland carcinoma, kidney cancer, liver cancer, prostate cancer, vulval cancer, thyroid cancer, hepatic carcinoma, and head and neck cancer. I

22. The method of items 15-21, wherein the pancreatic cancer is selected from the group consisting of: primary pancreatic cancer, metastatic pancreatic cancer, refractory pancreatic cancer, cancer drug resistant pancreatic cancer and pancreatic ductal adenocarcinoma.

23. The method of items 15-22, wherein the cancer is pancreatic ductal adenocarcinoma.

24. A kit composition, or medicament comprising an iRGD-analog, an immune checkpoint inhibitor, a nucleoside; and a taxane.

25. The kit, composition or medicament of item 24, wherein the iRGD-analog is LSTA-1 (certepeptide; CAS Registry #: 2580154-02-3), the immune checkpoint inhibitor is durvalumab, the nucleoside is gemcitabine; and the taxane is nab-paclitaxel.

### BRIEF DESCRIPTION OF THE FIGURES

FIG. 1 shows a diagram corresponding to the iLSTA: Phase 1b/2a clinical trial in locally advanced PDAC with chemotherapy & Immuno-Oncology.

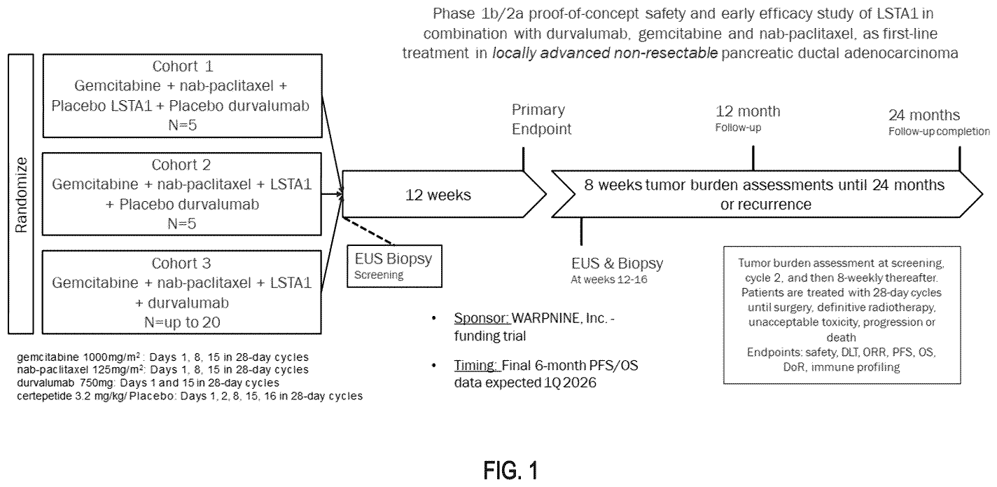

FIG. 2 shows the Preliminary Efficacy Results of the iLSTA clinical trial.

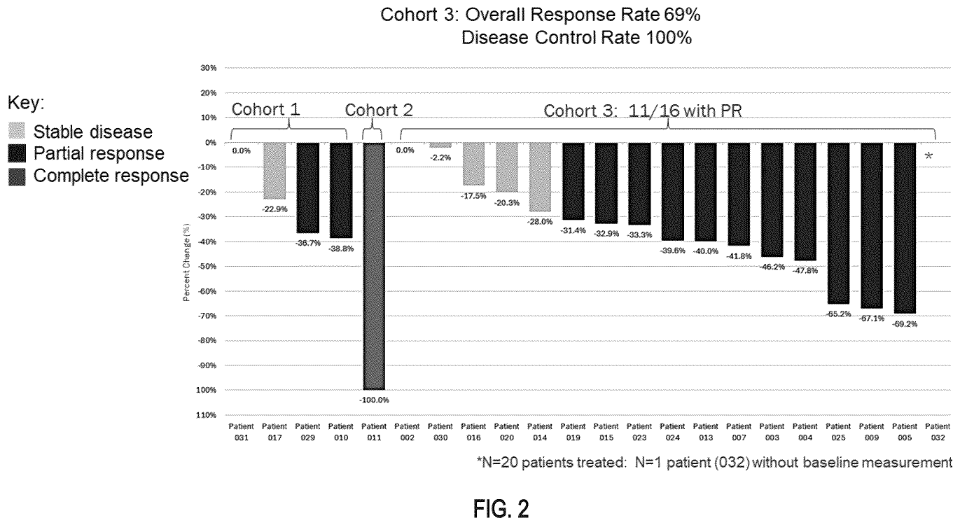

### DETAILED DESCRIPTION

Provided herein are methods of treating pancreatic cancer in a patient in need thereof, comprising administering to the patient an effective amount of LSTA-1, or pharmaceutically acceptable salt thereof, in combination with anti-cancer therapy comprising durvalumab, gemcitabine and nab-paclitaxel, or pharmaceutically acceptable salts thereof. In particular embodiments, the pancreatic cancer is selected from the group consisting of: primary pancreatic cancer, metastatic pancreatic cancer, refractory pancreatic cancer, cancer drug resistant pancreatic cancer and pancreatic ductal adenocarcinoma (PDAC). In a particular embodiment, the cancer is pancreatic ductal adenocarcinoma (PDAC) (Stage 0-IV).

Accordingly, the invention provides improved methods and medicaments for more effectively treating solid tumors with immune checkpoint inhibitors. LSTA-1 (also referred to herein as certepepfide) is a tumor-penetrating peptide that is an analog of iRGD (internalizing arginylglycylaspartic acid cyclic peptide). iRGD peptide molecules in general, and LSTA-1 in particular as an iRGD-analog, have a cyclizing (S—S bond through the cysteine side chains) structure containing nine amino acids. The pharmacological effect of iRGD peptides, including LSTA-1 is restricted to tumors via the primary RGD tumor homing motif interaction with αv-integrins (highly expressed in growing tumors but not in healthy tissues). The secondary ‘CendR’—motif modulates the tumor microenvironment via NRP-1. Based on experimental models, the interaction with neuropilin-1 leads to transformation of the solid tumor microenvironment into a temporary drug conduit, allowing an efficient tumor access of durvalumab and other immune checkpoint inhibitors given in combination with iRGD, such as LSTA-1, and the like. Studies have demonstrated that LSTA-1 increases, via the above-mentioned tumor microenvironment modulation mechanism, accumulation and penetration of anticancer drugs into tumors, but not into normal tissues. As a result, anti-tumor activity is enhanced, while the therapeutic margins/safety profile is potentially improved.

As used herein, the term “iRGD” or “iRGD peptide” or “iRGD analog” refers to a 9-amino acid cyclic peptide having sequence (sequence: CRGDKGPDC; SEQ ID NO: 1) or a variant thereof. In certain specific examples, variants of iRGD include the following CRGD(R/K/H)G(P/V)(D/E/H) C (SEQ ID NO:2), wherein the parentheses set forth amino acid options at that position. Other iRGD variants are disclosed in U.S. Pat. Nos. 8,367,621; and 9,115,170, which is incorporated herein in its entirety. Reference to iRGD, iRGD peptide or iRGD analog includes peptide variants unless stated otherwise.

A particular iRGD-analog for use herein corresponds to LSTA-1 (also referred to herein as certepeptide) and has the following molecular formula C37 H60 N14 O14 S2; a MW 989.1; and corresponds to CAS Registry #: 2580154-02-3. Another iRGD for use herein has the Molecular Formula: C₃₅H₅₇N₁₃O₁₄S₂; a Molecular Weight of 948.04; and CAS Registry No. 1392278-76-0, can be used in the invention methods, in view of the data, dosages and results provided herein. The structure of LSTA-1 (certepeptide) corresponds to:

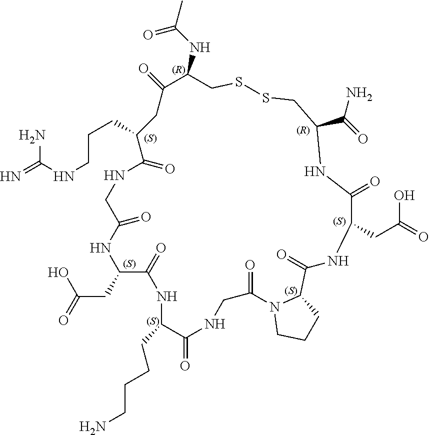

As used herein, the term “immune checkpoint inhibitor” refers to a class of agents that activate the immune system to attack tumors by blocking or reducing the activity of immune checkpoint molecules such as CTLA4, PD-1, PD-L1, and the like, discussed below.

An important function of the immune system is its ability to tell between normal cells in the body and those it sees as “foreign.” This lets the immune system attack the foreign cells while leaving the normal cells alone. To do this, it uses “checkpoints.” Immune checkpoints are molecules on certain immune cells that need to be activated (or inactivated) to start an immune response. In summary, immune checkpoints are immune system regulators that are crucial for self tolerance, which prevents the immune system from attacking normal cells. However, some tumors can protect themselves from attack by the immune system by manipulating this system. Drugs that target these checkpoints are called checkpoint inhibitors.

Immune checkpoint molecules can be stimulatory (e.g., members of the tumor necrosis factor receptor superfamily such as CD27, CD40, OS40, GITR, and CD137) or inhibitory (e.g., A2AR, B7-H3, B7-H4, BTLA, CTLA4, IDO, KIR, LAG3, NOX2, PD-1, TIM-3, VISTA, and SIGLEC7).

As set forth above, immune checkpoint inhibitors are molecules (drugs) that inhibit or block inhibition of the immune system, such as by blocking inhibitory checkpoint proteins. Examples of checkpoint proteins include CTLA4, and/or PD-1, and/or PD-L1 and/or PD-L2. Pembrolizumab (lambrolizumb; Keytruda), Nivolumab (Opdivo), Atezolizumab (Tecentriq), Avelumab (Bevancio), cemiplimab (Libtayo), and Durvalumab (Imfinzi) are FDA-approved drugs that inhibit PD-1/PD-L1, and are contemplated for use with the invention. Additional immune checkpoint inhibitors include MEDI0680, MPDL3280A, AMP-224, BMS-936559, MPDL3280A, MEDI4736, MSB0010718C, for example. The synergistic effects of with immune checkpoint inhibitors should apply to any of the foregoing immune checkpoint inhibitors, or newly developed immune checkpoint inhibitors targeting the aforementioned inhibitory checkpoint proteins or other inhibitory checkpoint proteins to be elucidated.

Preferred immune checkpoint inhibitors are monoclonal antibodies that target either PD-1 or PD-L1 to block this binding and boost the immune response against cells. Exemplary drugs that target and antagonize or block PD-1 include: pembrolizumab (Keytruda); nivoluniab (Opdivo); and cemiplirnab (Libtayo). Exemplary drugs that target and block or antagonize PD-L1 include: atezolizumab (Tecentriq); avelumab (Bavencio); and durvalumab (Imfinzi). These drugs can be helpful treating several types of cancer.

As used herein, the terms “treatment,” “treating,” and the like, refer to obtaining a desired pharmacologic and/or physiologic effect through administering compound(s) or composition(s). “Treatment,” includes: preventing, partially preventing, reversing, alleviating, reducing the likelihood of, or inhibiting the condition or disease (or symptom thereof) from occurring in a subject. The subject can include those diagnosed with a tumor or cancer, a pre-cancer, or who are predisposed to the condition or disease but has not yet been diagnosed as having it; (b) inhibiting the condition or disease or symptom thereof, such as, arresting its development; and (c) relieving, alleviating or ameliorating the condition or disease or symptom thereof, such as, for example, causing regression of the condition or disease or symptom thereof. Treatment can include administering one or more agents, performing a procedure such as surgery or applying radiation and the like, or both.

As used herein, the term “administering” and its cognates refer to introducing an agent to a subject, and can be performed using any of the various methods or delivery systems for administering agents or pharmaceutical compositions, and any route suitable for the composition and the subject, as known to those skilled in the art. Modes of administering include, but are not limited to oral administration, intravenous, subcutaneous, intramuscular or intraperitoneal injections, or local administration directly into or onto a target tissue (such as the pancreas, brain, or a tumor). Administration by any route or method that delivers a therapeutically effective amount of the drug or composition to the cells or tissue to which it is targeted is suitable for use with the invention.

As used herein, the term “combination,” with respect to administration of more than one active agent to a subject, i.e., combination therapy, refers to administration simultaneously or at different times. The one or more agents can be delivered in two or several pharmaceutical compositions that contain one active agent each, or using pharmaceutical compositions that each contain one or more active agent(s). The different pharmaceutical compositions can be formulated for the same or different routes of administration. The administration of the separate pharmaceutical compositions can be accomplished at the same time, in quick succession, or separated in time by minutes, hours, days, or weeks. A combination pharmaceutical composition contains more than one active agent and a pharmaceutically acceptable carrier.

As used herein, the terms “subject,” “individual,” “host,” and “patient,” are used interchangeably to refer to humans or any non-human mammal, and can include mammalian farm animals, mammalian sport animals, mammalian companion animals, simians, non-human primates, felines, canines, equines, rodents, lagomorphs, bovines, porcines, ovines, caprines. A suitable subject for the invention preferably is a human that is suspected of having, has been diagnosed as having, or is at risk of developing a hyperproliferative disease. Conditions amenable to treatment by the invention which define an appropriate subject or patient will be discerned easily by the person of skill in the art based on the disclosures herein. A “subject in need” is a subject that is at risk of developing cancer, or who manifests any characteristics or symptoms of cancer, or who has been diagnosed with cancer.

As used herein, the term “cancer”, also referred to as a tumor or a malignant tumor, refers to any of a group of diseases involving abnormal cell proliferation (hyperproliferation) with the potential to invade locally and/or spread to other parts of the body (metastasize). The term “cancer” is generally used interchangeably with “tumor” herein (unless a tumor is specifically referred to as a “benign” tumor, which is an abnormal mass of cells that lacks the ability to invade neighboring tissue or metastasize), and encompasses malignant solid tumors (e.g., carcinomas, sarcomas) and malignant growths in which there may be no detectable solid tumor mass (e.g., certain hematologic malignancies). In particular, cancers that are susceptible to immune checkpoint inhibitors are contemplated for use with the methods according to the invention, however immune checkpoint inhibitor-resistant cancers also can be treated according to embodiments of the invention. The term “cancer” can refer to a primary or metastatic tumor, and includes cancers that are unresectable cancer, and cancers of any stage, including stage III cancer and/or stage IV cancer.

All combinations of any genus, subgenus, or species of immune checkpoint inhibitor and any genus, subgenus, or species of complement inhibitor, compositions comprising any such combination, and use of any such combination in any method described herein, are to be considered expressly disclosed herein. Any antibody or other specific binder that can block or inhibit CTL-4, and/or PD-1 and/or PD-L1 and/or PD-L2 can be used with the inventive methods, such as nanoparticles, engineered cells, any engineered binding protein, soluble receptor, aptamer, peptide or small molecule that binds to an immune checkpoint protein and preferably antagonizes or blocks an inhibitory immune checkpoint molecule. Combinations or mixtures of any of such immune checkpoint inhibitors are suitable for use with the invention. In a particular embodiment, the immune checkpoint inhibitor for use herein is durvalumab.

In certain embodiments, the tumor is a malignant solid tumor characterized by dense tumor stroma. In other embodiments, the tumor is a solid tumor of a cancer selected from the group consisting of: breast cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colon cancer, colorectal cancer, endometrial carcinoma, salivary gland carcinoma, kidney cancer, liver cancer, prostate cancer, vulval cancer, thyroid cancer, hepatic carcinoma, and head and neck cancer. In another embodiment, the pancreatic cancer is selected from the group consisting of: primary pancreatic cancer, metastatic pancreatic cancer, refractory pancreatic cancer, cancer drug resistant pancreatic cancer and adenocarcinoma. In a particular embodiment, the cancer is ductal adenocarcinoma (such as Stage 0-IV, and the like).

As used herein the phrase “solid tumor” refers to essentially solid neoplasmic growth, with low liquid content that is other than a cyst or tumor metastasis (i.e. at its metastatic stage of disease).

As used herein, the phrase “in a combination” refers to administering more that one therapeutic agent to a respective patient in need thereof. In particular embodiments, LSTA-1 is administered with at least one other anti-cancer therapeutic agent.

As used herein, the phrase “simultaneous, separate or sequential administration” refers to administering LSTA-1 at the same time as the one or more other cancer therapeutic agents; or either before or after administration with the co-administered anti-cancer agents; such that the co-administration can be from separate pharmaceutical compositions administered with either the same or different dosing regimens. In certain embodiments, LSTA-1 is administered before the subsequent and sequential administration of the one or more anti-cancer agents.

As used herein, the term “malignant” refers to a tumor or cancer in which abnormal cells divide without control and can invade nearby tissues. Malignant cancer cells can also spread to other parts of the body through the blood and lymph systems.

In certain embodiments, the methods and medicaments of the present invention are suitable for using an iRGD peptide or LSTA-1 (certepeptide an iRGD-analog) to enhance the therapeutic effects of any immune checkpoint inhibitor, such as durvalumab and the like, used to treat solid tumors. The methods and medicaments of the present invention can thus contain combinations of an iRGD-analog (LSTA-1) with any immune checkpoint inhibitor, such as durvalumab and the like, used to treat solid tumors, with at least one of a taxane such as docetaxel or paclitaxel (including nab-paclitaxel), and a nucleoside such as gemcitabine. In a particular embodiment, the methods and medicaments of the present invention use a combination of LSTA-1 (certepeptide) with durvalumab, nab-paclitaxel, and gemcitabine for treating pancreatic cancer, such a pancreatic ductal adenocarcinoma (PDAC).

In particular embodiments, iRGD or LSTA-1 (also referred to as certepeptide) is administered in an amount selected from the group consisting of: about 0.2 to 20 mg/kg body weight/per dose of cancer therapy, about 0.3 to 17 mg/kg body weight/per dose of cancer therapy, about 0.4 to 14 mg/kg body weight/per dose of cancer therapy, about 0.5 to 11 mg/kg body weight/per dose of cancer therapy, about 0.6 to 8 mg/kg body weight/per dose of cancer therapy, about 0.7 to 5 mg/kg body weight/per dose of cancer therapy, about 0.8 to 3.2 mg/kg body weight/per dose of cancer therapy. In a particular embodiment, iRGD or LSTA-1 is administered in an amount corresponding to 3.2 mg/kg body weight/per dose of cancer therapy.

As used herein, the phrase “per dose of cancer therapy” refers to the co-administration of iRGD or LSTA-1 with one or more anti-cancer agents, such that each time an anti-cancer therapeutic is administered, iRGD or LSTA-1 is likewise co-administered to facilitate the therapeutic's penetration into the tumor. The co-administration per dose of iRGD or LSTA-1 does not need to be exactly simultaneous with the therapeutic agent(s), and iRGD or LSTA-1 can be administered either before or after the administration of the therapeutic agent.

In certain embodiments, iRGD or LSTA-1 is administered before or during the administration of anti-cancer therapy, wherein the cancer therapy is at a dosing regimen selected from the group consisting of: 4 times/day, 3 times/day, twice daily, once daily, once every other day, once every 2nd day, once every 3rd day, once every 4th day, once every 5th day, once every 6th day, once weekly, once every 8th day, once every 9th day, once every 10th day, once every 11th day, once every 12th day, once every 13th day, once every 2 weeks, once every 3 weeks, and/or once per month. In a particular embodiment, iRGD or LSTA-1 is administered on days 1, 2, 8, 15, 16 in 28-day cycles.

Accordingly, in a particular embodiment, 3.2 mg/kg body weight/per dose of LSTA-1 (certepeptide) is administered on days 1, 2, 8, 15, 16 in 28-day cycles.

In one embodiment, iRGD or LSTA-1 is present in a dry formulation or suspended in a biocompatible medium. In particular embodiments, the biocompatible media is selected from the group consisting of: water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, and lipid-containing emulsions. In a particular embodiment, iRGD or LSTA-1 is administered intravenously.

The method of the present invention is particularly suitable for the treatment of pancreatic cancer, which is characterized by a prominent dense tumor stroma, acting as a physical barrier to drug entry. As an example of clinical usefulness, provided herein are safety and efficacy results of LSTA-1 (certepeptide) when given alone or in combination with durvalumab, nab-paclitaxel and gemcitabine, including its ability to enhance tumor response.

Also provided herein is a method of treating pancreatic cancer in a patient in need thereof, comprising administering to the patient an effective amount of iRGD or LSTA-1, in combination with durvalumab, gemcitabine and nab-paclitaxel, or pharmaceutically acceptable salts thereof. In certain embodiments, the pancreatic cancer is selected from the group consisting of: primary pancreatic cancer, metastatic pancreatic cancer, refractory pancreatic cancer, cancer drug resistant pancreatic cancer and adenocarcinoma. In a particular embodiment, the cancer is pancreatic ductal adenocarcinoma (PDAC) (Stage 0-IV).

Also provided herein is a method of treating pancreatic cancer (e.g., PDAC), colon cancer or appendiceal cancer in a patient in need thereof, comprising administering to the patient an effective amount of iRGD or LSTA-1, in combination with durvalumab, gemcitabine and nab-paclitaxel, or pharmaceutically acceptable salts thereof.

In context of the present invention it was found that using an iRGD or LSTA-1 can enhance the clinical activity of other pancreatic cancer drugs such as durvalumab, gemcitabine and nab-paclitaxel administered by the intravenous route.

In certain embodiments, iRGD or LSTA-1 is administered in an amount selected from the group consisting of: about 0.2 to 20 mg/kg body weight/per dose of cancer therapy, about 0.3 to 17 mg/kg body weight/per dose of cancer therapy, about 0.4 to 14 mg/kg body weight/per dose of cancer therapy, about 0.5 to 11 mg/kg body weight/per dose of cancer therapy, about 0.6 to 8 mg/kg body weight/per dose of cancer therapy, about 0.7 to 5 mg/kg body weight/per dose of cancer therapy, about 0.8 to 3.2 mg/kg body weight/per dose of cancer therapy. In one embodiment, iRGD or LSTA-1 (certepeptide) is administered in an amount corresponding to 3.2 mg/kg body weight/per dose of cancer therapy.

In particular embodiments, the iRGD, e.g., LSTA-1 and the like, is administered before or during the administration of anti-cancer therapy, wherein the cancer therapy is at a dosing regimen selected from the group consisting of: 4 times/day, 3 times/day, twice daily, once daily, once every other day, once every 2nd day, once every 3rd day, once every 4th day, once every 5th day, once every 6th day, once weekly, once every 8th day, once every 9th day, once every 10th day, once every 11th day, once every 12th day, once every 13th day, once every 2 weeks, once every 3 weeks, and/or once per month. In a particular embodiment, iRGD or LSTA-1 is administered on days 1, 2, 8, 15, 16 in 28-day cycles. Accordingly, in a particular embodiment, 3.2 mg/kg body weight/per dose of LSTA-1 (certepeptide) is administered on days 1, 2, 8, 15, 16 in 28-day cycles.

In a particular embodiment for treating pancreatic cancer,
LSTA-1 is administered in a range amount selected from: 0.01-100, 0.02-90, 0.03-80, 0.04-70, 0.05-60, 0.06-50, 0.07-40, 0.08-30, 0.09-30, 0.1-25, 0.11-20, 0.12-15, 0.13-10, 0.14-9, 0.15-8, 0.16-7, 0.17-6, 0.18-5, 0.19-4, or 0.2-3.2 mg/kg body weight/day or per dose of chemotherapy;
durvalumab is administered in a range amount selected from: 1-2000, 50-1900, 100-1800, 200-1700, 300-1600, 350-1500, 400-1400, 450-1300, 500-1200, 550-1100, 600-1000, 650-900, and 700-800 mg;
nab-paclitaxel is administered in a range amount selected from: 1-500, 10-450, 20-400, 30-350, 40-300, 50-250, 60-200, 70-175, 80-160, 90-150, 100-140, 110-140, 115-135 or 120-130 mg/m²; and
gemcitabine is administered in a range amount selected from: 1-5000, 100-4500, 200-4000, 300-3500, 400-3000, 500-2500, 550-2000, 600-1750, 650-1500, 700-1400, 750-1300, 800-1200, or 900-1100 mg/m².

In certain embodiments, LSTA-1 (certepeptide) is administered on days 1, 2, 8, 15, 16 in 28-day cycles; durvalumab is administered on days 1 and 15 in 28-day cycles; nab-paclitaxel is administered on days 1, 8, 15 in 28-day cycles; and gemcitabine is administered on days 1, 8, 15 in 28-day cycles.

In yet another embodiment for treating pancreatic cancer: LSTA-1 is administered in a range of 0.2-3.2 mg/kg body weight/day or per dose of chemotherapy; durvalumab is administered at 750 mg; nab-paclitaxel is administered at 125 mg/m²; and gemcitabine is administered at 1000 mg/m². In a particular embodiment for treating pancreatic ductal adenocarcinoma (PDAC), LSTA-1 (certepeptide) is administered at 3.2 mg/kg body weight/day or per dose on days 1, 2, 8, 15, 16 in 28-day cycles; durvalumab is administered at 750 mg on days 1 and 15 in 28-day cycles; nab-paclitaxel is administered at 125 mg/m² on days 1, 8, 15 in 28-day cycles; and gemcitabine is administered at 1000 mg/m² on days 1, 8, 15 in 28-day cycles. In certain embodiments, the dosing regimen is administered for at least 2 cycles, at least 4 cycles, at least 6 cycles, at least 8 cycles, at least 10 cycles of administration. In other embodiments, the dosing regimen is administered for: 2 cycles, 4 cycles, 6 cycles, 8 cycles, 10 cycles, or 12 cycles of administration. In a particular embodiment, the dosing regimen is administered for 6 cycles.

In certain embodiments, efficacy or clinical activity of the method is measured by determining RECIST response. In one embodiment after 2 treatment cycles, 6 of 21 evaluable patients showed significant RECIST partial response after 2 cycles of treatment (5 patients in cohort 3), with the remaining 15 patients presenting with stable disease.

In another embodiment after 4 cycles of treatment, 12/21 patients demonstrated partial response (10 patients in cohort 3); and of the remaining 9 patients, 7 demonstrated stable disease, and 1 patient (cohort 2) exhibited a phenomenal RECIST complete response. Sixteen of 21 patients who completed 4 treatment cycles showed a decrease in CA19-9 levels with 7 of those 16 patients demonstrating >90% reduction in CA19-9 (5 in cohort 3).

In yet another embodiment after 6 cycles of treatment, 12/20 patients demonstrated partial response (10 patients in cohort 3). Of the remaining 8 patients, 7 demonstrated stable disease and 1 patient (cohort 2) exhibited a continued RECIST complete response. Fifteen of 20 patients who completed 6 cycles of treatment showed a decrease in CA 19-9 levels and 8 of those 15 patients demonstrated >90% reduction in CA19-9 (6 in cohort 3).

Ten of 21 patients had baseline and post-treatment biopsies which could be analyzed for tumor infiltrating lymphocytes (9 from cohort 3), with 9 patients (8 from chart 3) showing significant immune cell infiltration (10% to 50% stroma infiltration). For 5 of 21 patients (3 in cohort 3) there were no tumor cells present in the post-treatment biopsy. For the remaining 6 of 21 patients, paired biopsies were not available at the time of analysis.

As used herein, the phrase “CA 19-9 levels” refers to the protein produced by cancer and other cells in the body that is used as a tumor marker to help detect pancreatic cancer. The normal CA 19-9 range in a healthy person is 0-37 units per milliliter. CA 19-9 levels can be higher in patients with pancreatic cancer. In general: Rising CA 19-9 values mean the tumor is growing.

As used herein, the phrase “tumor infiltrating lymphocytes (TILs)” refers to a type of immune cell that can recognize and kill cancer cells. They are white blood cells that patrol the body to identify abnormal cells, including cancer cells.

Also provided herein is a medicament or method for treating, inhibiting, or reducing the volume of a tumor of a cancer in a subject or patient in need thereof, wherein the method comprises administering an iRGD (e.g., certepeptide), or a pharmaceutically acceptable salt thereof, in a combination with simultaneous, separate or sequential administration of an anti-cancer therapy comprising an immune checkpoint inhibitor, a nucleoside; and a taxane.

In particular embodiments of the medicament or method, iRGD or LSTA-1 is administered in a range amount selected from: 0.01-100, 0.02-90, 0.03-80, 0.04-70, 0.05-60, 0.06-50, 0.07-40, 0.08-30, 0.09-30, 0.1-25, 0.11-20, 0.12-15, 0.13-10, 0.14-9, 0.15-8, 0.16-7, 0.17-6, 0.18-5, 0.19-4, and 0.2-3.2 mg/kg body weight/day or per dose; the immune checkpoint inhibitor is administered in a range amount selected from: 1-2000, 50-1900, 100-1800, 200-1700, 300-1600, 350-1500, 400-1400, 450-1300, 500-1200, 550-1100, 600-1000, 650-900, and 700-800 mg; the taxane is administered in a range amount selected from: 1-500, 10-450, 20-400, 30-350, 40-300, 50-250, 60-200, 70-175, 80-160, 90-150, 100-140, 110-140, 115-135 and 120-130 mg/m²; and the nucleoside is administered in a range amount selected from: 1-5000, 100-4500, 200-4000, 300-3500, 400-3000, 500-2500, 550-2000, 600-1750, 650-1500, 700-1400, 750-1300, 800-1200, and 900-1100 mg/m².

In certain embodiments of the medicament or method for treating cancer, the iRGD (e.g., certepeptide) is administered at 3.2 mg/kg body weight/day or per dose; the immune checkpoint inhibitor is administered at 750 mg; the taxanel is administered at 125 mg/m²; and the nucleoside is administered at 1000 mg/m². In a particular embodiment, the iRGD is certepeptide administered at 3.2 mg/kg body weight/day or per dose; the immune checkpoint inhibitor is durvalumab administered at 750 mg; the taxanel is nab-paclitaxel administered at 125 mg/m²; and the nucleoside is gemcitabine administered at 1000 mg/m².

In a particular embodiment of the medicament or method for treating pancreatic ductal adenocarcinoma (PDAC), certepeptide is administered at 3.2 mg/kg body weight/day or per dose on days 1, 2, 8, 15, 16 in 28-day cycles; durvalumab is administered at 750 mg on days 1 and 15 in 28-day cycles; nab-paclitaxel is administered at 125 mg/m² on days 1, 8, 15 in 28-day cycles; and gemcitabine is administered at 1000 mg/m² on days 1, 8, 15 in 28-day cycles. In certain embodiments, the dosing regimen is administered for at least 2 cycles, at least 4 cycles, at least 6 cycles, at least 8 cycles, at least 10 cycles of administration. In other embodiments, the dosing regimen is administered for: 2 cycles, 4 cycles, 6 cycles, 8 cycles, 10 cycles, or 12 cycles of administration. In a particular embodiment, the dosing regimen is administered for 6 cycles.

Also provided herein are pharmaceutical compositions comprising an iRGD-analog, an immune checkpoint inhibitor, a nucleoside; and a taxane; and pharmaceutically acceptable excipients. In particular embodiments, the immune checkpoint inhibitor is durvalumab, the nucleoside is gemcitabine; and the taxane is nab-paclitaxel. Pharmaceutically acceptable excipients are well-known in the art. The pharmaceutical compositions can be administered to an individual (such as human) via a bolus injection or an infusion, via various routes, including, for example, intravenous, intra-arterial, intraperitoneal, intrapulmonary, oral and inhalation, subcutaneous. In some embodiments, the composition is administered intravenously.

The formulations can be presented in unit-dose or multi-dose sealed containers, such as ampules and vials, and can be stored in a freeze-dried (lyophilized) condition requiring only the addition of the sterile liquid excipient, for example, saline, for injections, immediately prior to use.

In particular embodiments, LSTA-1 for injection is a sterile, white, lyophilized powder supplied as 100 mg per vial of active ingredient dose strength for intravenous administration. LSTA-1 Injection consists of LSTA-1 drug substance with sodium acetate trihydrate and mannitol as excipients.

Provided herein are medicaments or methods for treating, inhibiting, or reducing the volume of a tumor of a cancer in a subject or patient in need thereof, wherein the method comprises administering an iRGD-analog, or a pharmaceutically acceptable salt thereof, in a combination with simultaneous, separate or sequential administration of an anti-cancer therapy regimen comprising an immune checkpoint inhibitor, a nucleoside; and a taxane, or a pharmaceutically acceptable salt thereof. In certain embodiments, the tumor is a malignant solid tumor characterized by dense tumor stroma. In other embodiments, the tumor is a solid tumor of a cancer selected from the group consisting of: breast cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colon cancer, colorectal cancer, endometrial carcinoma, salivary gland carcinoma, kidney cancer, liver cancer, prostate cancer, vulval cancer, thyroid cancer, hepatic carcinoma, and head and neck cancer. In another embodiment, the pancreatic cancer is selected from the group consisting of: primary pancreatic cancer, metastatic pancreatic cancer, refractory pancreatic cancer, cancer drug resistant pancreatic cancer and adenocarcinoma. In a particular embodiment, the cancer is ductal adenocarcinoma, such as Stage 0-IV, and the like.

In certain embodiments of the invention methods provided herein, efficacy or clinical activity of the method is measured by determining: Overall Response Rate (ORR), Progression Free Survival (PFS) and/or Overall Survival (OS). In yet further embodiments, efficacy or clinical activity of the method is measured by determining one or more of: an Overall Response Rate (ORR) selected from greater than 25%, 30%, 35%, 40%, 45%, 50%, 55%, 60%, 65%, 70%, 75%, 80%, 85%, 90%, or greater that 95%; a Progression Free Survival (PFS) selected from greater than 25%, 30%, 35%, 40%, 45%, 50%, 55%, 60%, 65%, 70%, 75%, 80%, 85%, 90%, or greater that 95%; and/or an Overall Survival (OS) selected from greater than 25%, 30%, 35%, 40%, 45%, 50%, 55%, 60%, 65%, 70%, 75%, 80%, 85%, 90%, or greater that 95%.

In one embodiment, LSTA-1 is present in a dry formulation or suspended in a biocompatible medium. In other embodiments, the biocompatible media is selected from the group consisting of: water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, and lipid-containing emulsions. In a particular embodiment, LSTA-1 is administered intravenously.

Also provided herein is a kit, composition or medicament comprising an iRGD-analog, an immune checkpoint inhibitor, a nucleoside; and a taxane. In particular embodiments of the kit composition, or medicament, the iRGD-analog is LSTA-1 (CAS Registry #: 2580154-02-3), the immune checkpoint inhibitor is durvalumab, the nucleoside is gemcitabine; and the taxane is nab-paclitaxel.

### EXAMPLES

### Example 1—Immunotherapy Combined with an iRGD Peptide Plus Nab-Paclitaxel and Gemcitabine for Locally Advanced Pancreatic Ductal Adenocarcinoma

Pancreatic ductal adenocarcinoma (PDAC) is characterised by a dense, extracellular matrix-rich stroma, which creates a physical barrier against drug penetration. It has been found that the iRGD peptide, LSTA-1, can increase the penetration of anti-cancer drugs to tumours through selective targeting of tumour endothelial cell receptors. Additionally, LSTA-1 administration promotes CD8+ immune cell tumour infiltration, potentially enabling immunotherapy as a treatment modality.

The purpose of this study was to assess the safety and feasibility of the combination of gemcitabine/nab-paclitaxel with LSTA-1 and durvalumab, and to identify signals of immune response in pancreatic adenocarcinoma.

### Method:

Participants diagnosed with locally advanced PDAC were divided into 3 cohorts in a 1:1:4 ratio. Cohort 1 (n=5) received gemcitabine (1000 mg/m²), nab-paclitaxel (125 mg/m²), placebo LSTA-1 (3.2 mg/kg) and placebo durvalumab (750 mg). Cohort 2 (n=5) received gemcitabine, nab-paclitaxel, LSTA-1, and placebo durvalumab. Cohort 3 (n=20) received gemcitabine, nab-paclitaxel, LSTA-1 and durvalumab. LSTA-1 (certepeptide) was administered at 3.2 mg/kg body weight/day or per dose on days 1, 2, 8, 15, 16 in 28-day cycles; durvalumab was administered at 750 mg on days 1 and 15 in 28-day cycles; nab-paclitaxel was administered at 125 mg/m² on days 1, 8, 15 in 28-day cycles; and gemcitabine was administered at 1000 mg/m² on days 1, 8, 15 in 28-day cycles.

Tissue biopsies via endoscopic ultrasound were required pre-treatment and between weeks 12 and 16 for analysis of tumour infiltrating lymphocytes (TILs). Patient tumours were assessed every 8 weeks using radiological imaging according to RECIST v1.1, and serum CA19-9 levels were analysed monthly.

### Results:

The results are shown in Tables 1-6 below. From Tables 1-2, six of 21 evaluable patients showed significant RECIST partial response after 2 cycles of treatment (5 patients in cohort 3), with the remaining 15 patients presenting with stable disease.

From Tables 3-4, after 4 cycles of treatment, 12/21 patients demonstrated partial response (10 patients in cohort 3). Of the remaining 9 patients, 7 demonstrated stable disease, and 1 patient (cohort 2) exhibited a phenomenal RECIST complete response. Sixteen of 21 patients who completed 4 treatment cycles showed a decrease in CA19-9 levels with 7 of those 16 patients demonstrating >90% reduction in CA19-9 (5 in cohort 3).

From Tables 5-6, after 6 cycles of treatment, 12/20 patients demonstrated partial response (10 patients in cohort 3). Of the remaining 8 patients, 7 demonstrated stable disease and 1 patient (cohort 2) exhibited a continued RECIST complete response. Fifteen of 20 patients who completed 6 cycles of treatment showed a decrease in CA 19-9 levels and 8 of those 15 patients demonstrated >90% reduction in CA19-9 (6 in cohort 3).

Ten of 21 patients had baseline and post-treatment biopsies which could be analyzed for tumor infiltrating lymphocytes (9 from cohort 3), with 9 patients (8 from chart 3) showing significant immune cell infiltration (10% to 50% stroma infiltration). For 5 of 21 patients (3 in cohort 3) there were no tumor cells present in the post-treatment biopsy. For the remaining 6 of 21 patients, paired biopsies were not available at the time of analysis.

### CONCLUSION

The results of this RECIST study demonstrate that the combination of gemcitabine and nab-paclitaxel with LSTA-1 and durvalumab, in patients in need thereof, is effective to generate a complete response, partial response or stable disease response in various patients; and to generate a significant tumour infiltrating lymphocyte response within the pancreatic tumour in various patients.

TABLE 1

| | | | Percentage |
|---|---|---|---|
| Participant | Baseline CA19-9 | Cycle 2 CA19-9 | Change |
| ID | Level (U/mL) | Level (U/mL) | in CA19-9 Level |
| Cohort 1 | | | |
| Patient 010 | 8572 | 5649 | −34.1% |
| Patient 017 | 23 | 15 | −34.8% |
| Patient 029 | 329 | 411 | 124.9% |
| Patient 031 | <2 | 3 | 150.0% |
| Cohort 2 | | | |
| Patient 011 | 42 | 19 | −54.8% |
| Cohort 3 | | | |
| Patient 002 | 163 | 290 | 177.9% |
| Patient 003 | 251 | 61 | −75.7% |
| Patient 004 | 8435 | 6675 | −20.9% |
| Patient 005 | <2 | 1 | <−50.0% |
| Patient 007 | 30208 | 7835 | −74.1% |
| Patient 009 | 30 | 27 | −10.0% |
| Patient 013 | 1567 | 169 | −89.2% |
| Patient 014 | 3620 | 1102 | −69.6% |
| Patient 015 | 409 | 273 | −33.3% |
| Patient 016 | 3008 | 1786 | −40.6% |
| Patient 019 | 3 | 6 | 150.0.7% |
| Patient 020 | 8 | 13 | 162.5% |
| Patient 023 | 1347 | 252 | −81.3% |
| Patient 024 | 3106 | 2059 | −33.7% |
| Patient 025 | 54 | 12 | −77.8% |
| Patient 030 | 2651 | 3727 | 140.6% |
| Patient 032 | 424 | 254 | −40.1% |

TABLE 2

| | Baseline CT | Cycle 2 CT | | |
|---|---|---|---|---|
| | Tumour | Tumour | Percentage Change | |
| Participant ID | Measurement | Measurement | in Tumour Size | RECIST Criteria |
| Cohort 1 | | | | |
| Patient 010 | 49 | 37 | −24.5% | Stable Disease |
| Patient 017 | 35 | 30 | −14.3% | Stable Disease |
| Patient 029 | 38 | 31 | −18.5% | Stable Disease |
| Patient 031 | 20 | 20 | 0.0% | Stable Disease |
| Cohort 2 | | | | |
| Patient 011 | 27 | 18 | −33.3% | Partial Response |
| Cohort 3 | | | | |
| Patient 002 | 28 | 28 | 0.0% | Stable Disease |
| Patient 003 | 26 | 26 | 0.0% | Stable Disease |
| Patient 004 | 23 | 22 | −4.3% | Stable Disease |
| Patient 005 | 39 | 27 | −30.8% | Partial Response |
| Patient 007 | 55 | 31 | −43.6% | Partial Response |
| Patient 009 | 73 | 43 | −41.1% | Partial Response |
| Patient 013 | 55 | 40 | −427.3% | Stable Disease |
| Patient 014 | 50 | 48 | −4.0% | Stable Disease |
| Patient 015 | 76 | 67 | −11.8% | Stable Disease |
| Patient 016 | 40 | 37 | −7.5% | Stable Disease |
| Patient 019 | 35 | 29 | −17.1% | Stable Disease |
| Patient 020 | 64 | 56 | −12.5% | Stable Disease |
| Patient 023 | 42 | 28 | −33.3% | Partial Response |
| Patient 024 | 43 | 32 | −25.6% | Stable Disease |
| Patient 025 | 46 | 25 | −45.7% | Partial Response |
| Patient 030 | 46 | 46 | 0.0% | Stable Disease |
| Patient 032 | — | — | — | N/A |

TABLE 3

| | | | Percentage |
|---|---|---|---|
| Participant | Baseline CA19-9 | Cycle 4 CA19-9 | Change |
| ID | Level (U/mL) | Level (U/mL) | in CA19-9 Level |
| Cohort 1 | | | |
| Patient 010 | 8572 | 411 | −95.2% |
| Patient 017 | 23 | 7 | −69.6% |
| Patient 029 | 329 | 31 | −90.6% |
| Patient 031 | <2 | 8 | 400.0% |
| Cohort 2 | | | |
| Patient 011 | 42 | 19 | −54.8% |
| Cohort 3 | | | |
| Patient 002 | 163 | 205 | 125.8% |
| Patient 003 | 251 | 96 | −61.8% |
| Patient 004 | 8435 | 1625 | −80.7% |
| Patient 005 | <2 | 4 | >50.0% |
| Patient 007 | 30208 | 137 | −99.5% |
| Patient 009 | 30 | 12 | −60.0% |
| Patient 013 | 1567 | 28 | −98.2% |
| Patient 014 | 3620 | 1486 | −59.0% |
| Patient 015 | 409 | 112 | −72.6% |
| Patient 016 | 3008 | 230 | −92.4% |
| Patient 019 | 3 | 32 | 1066.7% |
| Patient 020 | 8 | 13 | 162.5% |
| Patient 023 | 1347 | 27 | −98.0% |
| Patient 024 | 3106 | 167 | −94.6% |
| Patient 025 | 54 | 14 | −74.1% |
| Patient 030 | 2651 | 1425 | −46.2% |
| Patient 032 | 424 | — | — |

TABLE 4

| | Baseline CT | Cycle 4 CT | | |
|---|---|---|---|---|
| | Tumour | Tumour | Percentage Change | |
| Participant ID | Measurement | Measurement | in Tumour Size | RECIST Criteria |
| Cohort 1 | | | | |
| Patient 010 | 49 | 30 | −38.8% | Partial Response |
| Patient 017 | 35 | 27 | −22.9% | Stable Disease |
| Patient 029 | 38 | 24 | −36.7% | Partial Response |
| Patient 031 | 20 | — | — | — |
| Cohort 2 | | | | |
| Patient 011 | 27 | 0 | −100.0% | Complete Response |
| Cohort 3 | | | | |
| Patient 002 | 28 | 28 | 0.0% | Stable Disease |
| Patient 003 | 26 | 16 | −38.5% | Partial Response |
| Patient 004 | 23 | 17 | −26.1% | Stable Disease |
| Patient 005 | 39 | 14 | −64.1% | Partial Response |
| Patient 007 | 55 | 32 | −41.8% | Partial Response |
| Patient 009 | 73 | 30 | −58.9% | Partial Response |
| Patient 013 | 55 | 33 | −40.0% | Partial Response |
| Patient 014 | 50 | 39 | −22.0% | Stable Disease |
| Patient 015 | 76 | 51 | −32.9% | Partial Response |
| Patient 016 | 40 | 36 | −10.0% | Stable Disease |
| Patient 019 | 35 | 24 | −31.4% | Partial Response |
| Patient 020 | 64 | 51 | −20.3% | Stable Disease |
| Patient 023 | 42 | 28 | −33.3% | Partial Response |
| Patient 024 | 43 | 26 | −39.6% | Partial Response |
| Patient 025 | 46 | 24 | −47.8% | Partial Response |
| Patient 030 | 46 | 45 | −2.2% | Stable Disease |
| Patient 032 | — | — | — | — |

TABLE 5

| | Baseline | Cycle 6 | Percentage |
|---|---|---|---|
| | CA19-9 | CA19-9 | Change |
| | Level | Level | in CA19-9 |
| Participant ID | (U/mL) | (U/mL) | Level |
| Cohort 1 | | | |
| Patient 010 | 8572 | 541 | −93.7% |
| Patient 017 | 23 | 10 | −56.5% |
| Patient 029 | 329 | 22 | −93.3% |
| Patient 031 | <2 | 2 | 0.0% |
| Cohort 2 | | | |
| Patient 011 | 42 | 15 | −64.3% |
| Cohort 3 | | | |
| Patient 002 | 163 | 495 | 303.7% |
| Patient 003 | 251 | 1259 | 501.6% |
| Patient 004 | 8435 | 213 | −97.5% |
| Patient 005 | <2 | <2 | 0.0% |
| Patient 007 | 30208 | 220 | −99.3% |
| Patient 009 | 30 | 14 | −53.3% |
| Patient 013 | 1567 | 17 | −98.9% |
| Patient 014 | 3620 | 4203 | 116.1% |
| Patient 015 | 409 | 81 | −80.2% |
| Patient 016 | 3008 | 90 | −97.0% |
| Patient 019 | 3 | 6 | 200.0% |
| Patient 020 | 8 | 6 | −25.0% |
| Patient 023 | 1347 | 13 | −99.0% |
| Patient 024 | 3106 | 151 | −95.1% |
| Patient 025 | 54 | 12 | −77.8% |
| Patient 030 | 2651 | 560 | −78.9% |
| Patient 032 | 424 | — | — |

TABLE 6

| | Baseline CT | Cycle 6 CT | Percentage | |
|---|---|---|---|---|
| Participant | Tumour | Tumour | Change in | |
| ID | Measurement | Measurement | Tumour Size | RECIST Criteria |
| Cohort 1 | | | | |
| Patient 010 | 49 | 30 | −38.8% | Partial Response (C4) |
| Patient 017 | 35 | 28 | −22.9% | Stable Disease |
| Patient 029 | 38 | 24 | −36.7% | Partial Response (C4) |
| Patient 031 | 20 | — | — | — |
| Cohort 2 | | | | |
| Patient 011 | 27 | 0 | −100.0% | Complete Response |
| Cohort 3 | | | | |
| Patient 002 | 28 | 28 | 0.0% | Stable Disease (C4) |
| Patient 003 | 26 | 14 | −46.2% | Partial Response |
| Patient 004 | 23 | 12 | −47.8% | Partial Response |
| Patient 005 | 39 | 12 | −69.2% | Partial Response |
| Patient 007 | 55 | 27 | −41.8% | Partial Response |
| Patient 009 | 73 | 12 | −67.1% | Partial Response |
| Patient 013 | 55 | 44 | −20.0% | Stable Disease |
| Patient 014 | 50 | 36 | −28.0% | Stable Disease |
| Patient 015 | 76 | 51 | −32.9% | Partial Response |
| Patient 016 | 40 | 36 | −17.5% | Stable Disease |
| Patient 019 | 35 | 33 | −31.4% | Partial Response |
| Patient 020 | 64 | 51 | −15.6% | Stable Disease |
| Patient 023 | 42 | 28 | −33.3% | Partial Response |
| Patient 024 | 43 | 26 | −39.6% | Partial Response (C4) |
| Patient 025 | 46 | 24 | −65.2% | Partial Response (C4) |
| Patient 030 | 46 | 45 | −2.2% | Stable Disease (C4) |
| Patient 032 | — | — | — | — |

1. A method of treating pancreatic cancer in a patient in need thereof, comprising administering to the patient an effective amount of LSTA-1, or pharmaceutically acceptable salt thereof, in combination with anti-cancer therapy comprising durvalumab, gemcitabine and nab-paclitaxel, or pharmaceutically acceptable salts thereof.

2. The method of claim 1, wherein the pancreatic cancer is selected from the group consisting of: primary pancreatic cancer, metastatic pancreatic cancer, refractory pancreatic cancer, cancer drug resistant pancreatic cancer and pancreatic ductal adenocarcinoma (PDAC).

3. The method of claim 1, wherein the cancer is pancreatic ductal adenocarcinoma (Stage 0-IV).

4. The method of claim 1, wherein LSTA-1 is administered in an amount selected from the group consisting of: about 0.2 to 20 mg/kg body weight/per dose of anti-cancer therapy, about 0.3 to 17 mg/kg body weight/per dose of anti-cancer therapy, about 0.4 to 14 mg/kg body weight/per dose of anti-cancer therapy, about 0.5 to 11 mg/kg body weight/per dose of anti-cancer therapy, about 0.6 to 8 mg/kg body weight/per dose of anti-cancer therapy, about 0.7 to 5 mg/kg body weight/per dose of anti-cancer therapy, about 0.8 to 3.2 mg/kg body weight/per dose of anti-cancer therapy.

5. The method of claim 1, wherein LSTA-1 is administered in an amount corresponding to 3.2 mg/kg body weight/per dose.

6. The method of claim 1, wherein LSTA-1 is administered before or during the administration of anti-cancer therapy, wherein the anti-cancer therapy is at a dosing regimen selected from the group consisting of: 4 times/day, 3 times/day, twice daily, once daily, once every other day, once every 2nd day, once every 3rd day, once every 4th day, once every 5th day, once every 6th day, once weekly, once every 8th day, once every 9th day, once every 10th day, once every 11th day, once every 12th day, once every 13th day, once every 2 weeks, once every 3 weeks, and once per month.

7. The method of claim 1, wherein:
LSTA-1 is administered in a range amount selected from: 0.01-100, 0.02-90, 0.03-80, 0.04-70, 0.05-60, 0.06-50, 0.07-40, 0.08-30, 0.09-30, 0.1-25, 0.11-20, 0.12-15, 0.13-10, 0.14-9, 0.15-8, 0.16-7, 0.17-6, 0.18-5, 0.19-4, and 0.2-3.2 mg/kg body weight/day or per dose;
durvalumab is administered in a range amount selected from: 1-2000, 50-1900, 100-1800, 200-1700, 300-1600, 350-1500, 400-1400, 450-1300, 500-1200, 550-1100, 600-1000, 650-900, and 700-800 mg;
nab-paclitaxel is administered in a range amount selected from: 1-500, 10-450, 20-400, 30-350, 40-300, 50-250, 60-200, 70-175, 80-160, 90-150, 100-140, 110-140, 115-135 and 120-130 mg/m²; and
gemcitabine is administered in a range amount selected from: 1-5000, 100-4500, 200-4000, 300-3500, 400-3000, 500-2500, 550-2000, 600-1750, 650-1500, 700-1400, 750-1300, 800-1200, and 900-1100 mg/m².

8. The method of claim 1, wherein LSTA-1 is administered at 3.2 mg/kg body weight/day or per dose; durvalumab is administered at 750 mg; nab-paclitaxel is administered at 125 mg/m²; and gemcitabine is administered at 1000 mg/m².

9. The method of claim 1, wherein LSTA-1 (certepeptide) is administered at 3.2 mg/kg body weight/day or per dose on days 1, 2, 8, 15, 16 in 28-day cycles; durvalumab is administered at 750 mg on days 1 and 15 in 28-day cycles; nab-paclitaxel is administered at 125 mg/m² on days 1, 8, 15 in 28-day cycles; and gemcitabine is administered at 1000 mg/m² on days 1, 8, 15 in 28-day cycles.

10. The method of claim 9, wherein the dosing regimen is administered for at least 2 cycles, at least 4 cycles, at least 6 cycles, at least 8 cycles, at least 10 cycles of administration; or for: 2 cycles, 4 cycles, 6 cycles, 8 cycles, 10 cycles, or 12 cycles of administration.

11. The method of claim 10, the dosing regimen is administered for 6 cycles.

12. The method of claim 1, wherein efficacy or clinical activity of the method is measured by determining RECIST response, wherein after 6 treatment cycles: 12/20 patients demonstrated partial response; 7 patients demonstrated stable disease; and 1 patient demonstrated complete response.

13. The method of claim 1, wherein efficacy or clinical activity of the method is measured by determining CA19-9 levels, wherein after 6 treatment cycles: 15/20 patients demonstrated a decrease in CA19-9 levels, and 8 of these 15 patients demonstrated >90% decrease in CA19-9 levels.

14. The method of claim 1, wherein efficacy or clinical activity of the method is measured by determining infiltration of TILs into tumor stroma, wherein wherein after 6 treatment cycles: 9/10 patients biopsied demonstrated 15%-50% stroma infiltration by TILs.

15. A method for treating, inhibiting, or reducing the volume of a tumor of a cancer in a subject or patient in need thereof, wherein the method comprises administering a dosing regimen of an iRGD, or a pharmaceutically acceptable salt thereof, in a combination with simultaneous, separate or sequential administration of an anti-cancer therapy comprising an immune checkpoint inhibitor, a nucleoside; and a taxane.

16. The method of claim 15, wherein:
iRGD is administered in a range amount selected from: 0.01-100, 0.02-90, 0.03-80, 0.04-70, 0.05-60, 0.06-50, 0.07-40, 0.08-30, 0.09-30, 0.1-25, 0.11-20, 0.12-15, 0.13-10, 0.14-9, 0.15-8, 0.16-7, 0.17-6, 0.18-5, 0.19-4, and 0.2-3.2 mg/kg body weight/day or per dose;
the immune checkpoint inhibitor is administered in a range amount selected from: 1-2000, 50-1900, 100-1800, 200-1700, 300-1600, 350-1500, 400-1400, 450-1300, 500-1200, 550-1100, 600-1000, 650-900, and 700-800 mg;
the taxane is administered in a range amount selected from: 1-500, 10-450, 20-400, 30-350, 40-300, 50-250, 60-200, 70-175, 80-160, 90-150, 100-140, 110-140, 115-135 and 120-130 mg/m²; and
the nucleoside is administered in a range amount selected from: 1-5000, 100-4500, 200-4000, 300-3500, 400-3000, 500-2500, 550-2000, 600-1750, 650-1500, 700-1400, 750-1300, 800-1200, and 900-1100 mg/m².

17. The method of claim 15, wherein: the iRGD is administered at 3.2 mg/kg body weight/day or per dose; the immune checkpoint inhibitor is administered at 750 mg; the taxanel is administered at 125 mg/m²; and the nucleoside is administered at 1000 mg/m²; or wherein the iRGD is LSTA-1 (certepeptide) administered at 3.2 mg/kg body weight/day or per dose on days 1, 2, 8, 15, 16 in 28-day cycles; wherein the immune checkpoint inhibitor is durvalumab administered at 750 mg on days 1 and 15 in 28-day cycles; the taxanel is nab-paclitaxel administered at 125 mg/m² on days 1, 8, 15 in 28-day cycles; and wherein the nucleoside is gemcitabine administered at 1000 mg/m² on days 1, 8, 15 in 28-day cycles.

18. The method of claim 15, wherein the dosing regimen is administered for at least 2 cycles, at least 4 cycles, at least 6 cycles, at least 8 cycles, at least 10 cycles of administration; or for: 2 cycles, 4 cycles, 6 cycles, 8 cycles, 10 cycles, or 12 cycles of administration; or wherein the dosing regimen is administered for 6 cycles.

19. The method of claim 15, wherein the tumor is a solid tumor of a cancer selected from the group consisting of: breast cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colon cancer, colorectal cancer, endometrial carcinoma, salivary gland carcinoma, kidney cancer, liver cancer, prostate cancer, vulval cancer, thyroid cancer, hepatic carcinoma, and head and neck cancer; wherein the pancreatic cancer is selected from the group consisting of: primary pancreatic cancer, metastatic pancreatic cancer, refractory pancreatic cancer, cancer drug resistant pancreatic cancer and pancreatic ductal adenocarcinoma.

20. A kit composition, or medicament comprising LSTA-1 (certepeptide; CAS Registry #: 2580154-02-3), durvalumab, gemcitabine; and nab-paclitaxel.
